(19) United States
(12) Patent Application Publication
(10) Pub. No.: US 2008/0280939 A1
(43) Pub. Date: Nov. 13, 2008

(54) Combination of Organic Compounds

(76) Inventors: Siego Izumo, Brookline, MA (US); Margaret Forney Prescott, Morris Plains, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/596,943

(22) PCT Filed: May 13, 2005
(86) PCT No.: PCT/EP05/05286

§ 371 Date: Sep. 5, 2007

Related U.S. Application Data
(60) Provisional application No. 60/571,729, filed on May 17, 2004.
(60) Provisional application No. 60/600,567, filed on Aug. 11, 2004.

(51) Int. Cl. A61K 31/4353; A61P 9/00; A61P 13/12; A61P 19/02

(52) U.S. Cl. 514/291

(57) ABSTRACT

The invention relates to a pharmaceutical combinations comprising an AT1 receptor blocker or pharmaceutically acceptable salts thereof and a mTOR inhibiting agent, e.g. rapamycin or a rapamycin derivative, optionally in the presence of a pharmaceutically acceptable carrier.

The present invention relates to a combination, such as a combined preparation or pharmaceutical composition, respectively, comprising an AT1 receptor blocker or pharmaceutically acceptable salts thereof and a mTOR inhibiting agent, e.g. rapamycin or a rapamycin derivative, optionally in the presence of a pharmaceutically acceptable carrier for simultaneous, separate or sequential use, especially in the prevention, delay of progression or treatment of cardiac and renal related conditions or diseases and arthritis or rheumatic arthritis related conditions or diseases; the use of such combination for the preparation of a pharmaceutical preparation for the prevention, delay of progression or treatment of such conditions; a method of prevention, delay of progression or treatment of cardiac and renal related conditions or diseases and arthritis or rheumatic arthritis related conditions or diseases.

The present invention relates to pharmaceutical combinations or compositions comprising an AT1 receptor blocker or pharmaceutically acceptable salts thereof and a mTOR inhibiting agent, e.g. rapamycin or a rapamycin derivative, optionally in the presence of a pharmaceutically acceptable carrier and their uses in treating cardiac and renal related conditions or diseases and their uses in treating or preventing arthritis or rheumatic arthritis related conditions or diseases.

The present invention furthermore relates to pharmaceutical combinations or compositions which comprise in combination an AT 1-receptor blocker and a mTOR inhibiting agent, e.g. rapamycin or a rapamycin derivative selected from the group of: 32-deoxorapamycin, 16-pent-2-ynyloxy-32-deoxorapamycin, 16-pent-2-ynyloxy-32(S or R)-dihydro-rapamycin, 16-pent-2-ynyloxy-32(S or R)-dihydro-40-O-(2-hydroxyethyl)-rapamycin, 40-[3-hydroxy-2-(hydroxymethyl)-2-methylpropanoate]-rapamycin (also called CC1779), 40-epi-(tetrazolyl)-rapamycin (also called ABT578), 40-0-(2-hydroxyethyl)-rapamycin, 32-deoxorapamycin and 16-pent-2-ynyloxy-32(S)-dihydro-rapamycin and a pharmaceutically acceptable carrier for simultaneous, sequential or separate use.

In a preferred embodiment, the present invention relates to pharmaceutical combinations or compositions which comprise in combination the AT 1-receptor blocker valsartan or a pharmaceutically acceptable salt thereof and a mTOR inhibiting agent, e.g. rapamycin or a rapamycin derivative selected from the group of: 32-deoxorapamycin, 16-pent-2-ynyloxy-32-deoxorapamycin, 16-pent-2-ynyloxy-32(S or R)-dihydro-rapamycin, 16-pent-2-ynyloxy-32(S or R)-dihydro-40-O-(2-hydroxyethyl)-rapamycin, 40-[3-hydroxy-2-(hydroxymethyl)-2-methylpropanoate]-rapamycin (also called CC1779), 40-epi-(tetrazolyl)-rapamycin (also called ABT578),40-0-(2-hydroxyethyl)-rapamycin, 32-deoxorapamycin and 16-pent-2-ynyloxy-32(S)-dihydro-rapamycin and a pharmaceutically acceptable carrier for simultaneous, sequential or separate use.

In one aspect the present invention relates to pharmaceutical combinations or compositions according to the invention for the treatment or prevention of cardiac and renal related conditions or diseases and arthritis or rheumatic arthritis related conditions or diseases which comprise in combination an AT 1-receptor blocker, especially valsartan or a pharmaceutically acceptable salt thereof and rapamycin and a pharmaceutically acceptable carrier for simultaneous, sequential or separate use.

In one aspect the present invention relates to pharmaceutical combinations or compositions according to the invention for the treatment or prevention of cardiac and renal related conditions or diseases and arthritis or rheumatic arthritis related conditions or diseases which comprise in combination an AT 1-receptor blocker, especially valsartan or a pharmaceutically acceptable salt thereof and 32-deoxorapamycin and a pharmaceutically acceptable carrier for simultaneous, sequential or separate use.

In one aspect the present invention relates to pharmaceutical combinations or compositions according to the invention for the treatment or prevention of cardiac and renal related conditions or diseases and arthritis or rheumatic arthritis related conditions or diseases which comprise in combination an AT 1-receptor blocker, especially valsartan or a pharmaceutically acceptable salt thereof and 16-pent-2-ynyloxy-32-deoxorapamycin and a pharmaceutically acceptable carrier for simultaneous, sequential or separate use.

In one aspect the present invention relates to pharmaceutical combinations or compositions according to the invention for the treatment or prevention of cardiac and renal related conditions or diseases and arthritis or rheumatic arthritis related conditions or diseases which comprise in combination an AT 1-receptor blocker, especially valsartan or a pharmaceutically acceptable salt thereof and 16-pent-2-ynyloxy-32(S or R)-dihydro-rapamycin and a pharmaceutically acceptable carrier for simultaneous, sequential or separate use.

In one aspect the present invention relates to pharmaceutical combinations or compositions according to the invention for the treatment or prevention of cardiac and renal related conditions or diseases and arthritis or rheumatic arthritis related conditions or diseases which comprise in combination an AT 1-receptor blocker, especially valsartan or a pharmaceutically acceptable salt thereof and 16-pent-2-ynyloxy-32(S or R)-dihydro-40-O-(2-hydroxyethyl)-rapamycin and a pharmaceutically acceptable carrier for simultaneous, sequential or separate use.

In one aspect the present invention relates to pharmaceutical combinations or compositions according to the invention for the treatment or prevention of cardiac and renal related conditions or diseases and arthritis or rheumatic arthritis related conditions or diseases which comprise in combination an AT 1-receptor blocker, especially valsartan or a pharmaceutically acceptable salt thereof and 40-[3-hydroxy-2-(hydroxymethyl)-2-methylpropanoate]-rapamycin and a pharmaceutically acceptable carrier for simultaneous, sequential or separate use.

In one aspect the present invention relates to pharmaceutical combinations or compositions according to the invention for the treatment or prevention of cardiac and renal related conditions or diseases and arthritis or rheumatic arthritis related conditions or diseases which comprise in combination an AT 1-receptor blocker, especially valsartan or a pharmaceutically acceptable salt thereof and 40-epi-(tetrazolyl)-rapamycin and a pharmaceutically acceptable carrier for simultaneous, sequential or separate use.

In a preferred embodiment the present invention relates to pharmaceutical combinations or compositions according to the invention for the treatment or prevention of cardiac and renal related conditions or diseases and arthritis or rheumatic arthritis related conditions or diseases which comprise in combination an AT 1-receptor blocker, especially valsartan or a pharmaceutically acceptable salt thereof and 32-deoxorapamycin and a pharmaceutically acceptable carrier for simultaneous, sequential or separate use.

In another preferred embodiment the present invention relates to pharmaceutical combinations or compositions according to the invention for the treatment or prevention of cardiac and renal related conditions or diseases and arthritis or rheumatic arthritis related conditions or diseases which comprise in combination an AT 1-receptor blocker, especially valsartan or a pharmaceutically acceptable salt thereof and 16-pent-2-ynyloxy-32(S)-dihydro-rapamycin and a pharmaceutically acceptable carrier for simultaneous, sequential or separate use.

In a most preferred embodiment the present invention relates to pharmaceutical combinations or compositions according to the invention for the treatment or prevention of cardiac and renal related conditions or diseases and arthritis or rheumatic arthritis related conditions or diseases which comprise in combination an AT 1-receptor blocker, especially valsartan or a pharmaceutically acceptable salt thereof and 40-0-(2-hydroxyethyl)-rapamycin and a pharmaceutically acceptable carrier for simultaneous, sequential or separate use.

A further aspect of the present invention provides a pharmaceutical composition according to the invention, e.g, for the treatment or prevention of cardiac and renal related conditions or diseases, i e, selected from the group consisting of hypertension, heart failure such as (acute and chronic) congestive heart failure, left ventricular dysfunction and hypertrophic cardiomyopathy, diabetic cardiac myopathy, supraventricular and ventricular arrhythmias, atrial fibrillation, atrial flutter, detrimental vascular remodeling, myocardial infarction and its sequelae, atherosclerosis, angina (whether unstable or stable), renal insufficiency (diabetic and non-diabetic), angina pectoris, diabetes, secondary aldosteronism, primary and secondary pulmonary hypertension, renal failure conditions, such as diabetic nephropathy, glomerulonephritis, scleroderma, glomerular sclerosis, proteinuria of primary renal disease, and also renal vascular hypertension, diabetic retinopathy, the management of other vascular disorders, such as migraine, peripheral vascular disease, Raynaud's disease, luminal hyperplasia, cognitive dysfunction (such as Alzheimer's), glaucoma and stroke, and for the treatment or prevention of arthritis or rheumatic arthritis related conditions or diseases, i e, selected from the group consisting of, arthritis or rheumatic arthritis, e.g. including reduction of, alleviation of, stabilization of or relief from the symptoms or illness which affect the organism, particularly joints or vertebrae, including also slowing the progression (destruction of the joints) in moderate to severe rheumatoid arthritis, progressive, or erosive rheumatoid arthritis who had an inadequate response to treatment with disease-modifying antirheumatic drugs and the reduction of the side-effects (signs and symptoms) of arthritis or rheumatic arthritis comprising administering a therapeutically effective amount of combination of (i) an AT 1-receptor blocker or a pharmaceutically acceptable salt thereof and (ii) a mTOR inhibiting agent, e.g. rapamycin or a rapamycin derivative for example rapamycin or a rapamycin derivative selected from the group of: 32-deoxorapamycin, 16-pent-2-ynyloxy-32-deoxorapamycin, 16-pent-2-ynyloxy-32(S or R)-dihydro-rapamycin, 16-pent-2-ynyloxy-32(S or R)-dihydro-40-O-(2-hydroxyethyl)-rapamycin, 40-[3-hydroxy-2-(hydroxymethyl)-2-methylpropanoate]-rapamycin (also called CC1779), 40-epi-(tetrazolyl)-rapamycin (also called ABT578), 40-0-(2-hydroxyethyl) -rapamycin, 32-deoxorapamycin and 16-pent-2-ynyloxy-32(S)-dihydro-rapamycin and a pharmaceutically acceptable carrieror to a mammal in need of such treatment.

In this composition, components (i) and (ii) can be obtained and administered together, one after the other or separately in one combined unit dose form or in two separate unit dose forms. The unit dose form may also be a fixed combination.

Furthermore the invention provides the use of a pharmaceutical composition according to the invention for the preparation of a medicament for the treatment of cardiac and renal related conditions or diseases and arthritis or rheumatic arthritis related conditions or diseases.

In another embodiment, the invention provides the use of a pharmaceutical composition according to the invention for the preparation of a medicament for the treatment or prevention of cardiac and renal related conditions or diseases, i e, selected from the group consisting of hypertension, heart failure such as (acute and chronic) congestive heart failure, left ventricular dysfunction and hypertrophic cardiomyopathy, diabetic cardiac myopathy, supraventricular and ventricular arrhythmias, atrial fibrillation, atrial flutter, detrimental vascular remodeling, myocardial infarction and its sequelae, atherosclerosis, angina (whether unstable or stable), renal insufficiency (diabetic and non-diabetic), angina pectoris, diabetes, secondary aldosteronism, primary and secondary pulmonary hypertension, renal failure conditions, such as diabetic nephropathy, glomerulonephritis, scleroderma, glomerular sclerosis, proteinuria of primary renal disease, and also renal vascular hypertension, diabetic retinopathy, the management of other vascular disorders, such as migraine, peripheral vascular disease, Raynaud's disease, luminal hyperplasia, cognitive dysfunction (such as Alzheimer's), glaucoma and stroke, and for the treatment or prevention of arthritis or rheumatic arthritis related conditions or diseases, i e, selected from the group consisting of, arthritis or rheumatic arthritis, e.g. including reduction of, alleviation of, stabilization of or relief from the symptoms or illness which affect the organism, particularly joints or vertebrae, including also slowing the progression (destruction of the joints) in moderate to severe rheumatoid arthritis, progressive, or erosive rheumatoid arthritis who had an inadequate response to treatment with disease-modifying antirheumatic drugs and the reduction of the side-effects (signs and symptoms) of arthritis or rheumatic arthritis. In another embodiment, the invention provides the use of a pharmaceutical composition according to the invention for the treatment or prevention of hypertension, heart failure such as (acute and chronic) congestive heart failure.

The present invention provides a kit comprising in separate containers in a single package pharmaceutical combinations or compositions comprising in one container a pharmaceutical composition comprising a mTOR inhibiting agent, e.g. rapamycin or a rapamycin derivative,and in a second container a pharmaceutical composition comprising an AT 1-receptor blocker.

The present invention provides a kit comprising in separate containers in a single package pharmaceutical combinations or compositions comprising in one container a pharmaceutical composition comprising a mTOR inhibiting agent, e.g. rapamycin or a rapamycin derivative, and in a second container a pharmaceutical composition comprising the AT 1-receptor blocker valsartan.

The kit form is particularly advantageous when the separate components must be administered in different dosage forms or are administered at different dosage intervals.

The present invention relates to a package comprising an AT 1-receptor blocker (especially valsartan) together with instructions for use in combination with a mTOR inhibiting agent, e.g. rapamycin or a rapamycin derivative for the treatment or prevention of cardiac and renal related conditions or diseases.

In a preferred embodiment, the package according to the invention comprises in combination the AT 1-receptor blocker valsartan or a pharmaceutically acceptable salt thereof and rapamycin.

In another preferred embodiment, the package according to the invention comprises in combination the AT 1-receptor blocker valsartan or a pharmaceutically acceptable salt thereof and a rapamycin derivative selected from the group of: 32-deoxorapamycin, 16-pent-2-ynyloxy-32-deoxorapamycin, 16-pent-2-ynyloxy-32(S or R)-dihydro-rapamycin, 16-pent-2-ynyloxy-32(S or R)-dihydro-40-O-(2-hydroxyethyl)-rapamycin, 40-[3-hydroxy-2-(hydroxymethyl)-2-methylpropanoate]-rapamycin (also called CC1779), 40-epi-(tetrazolyl)-rapamycin (also called ABT578), 40-0-(2-hydroxyethyl) -rapamycin, 32-deoxorapamycin and 16-pent-2-ynyloxy-32(S)-dihydro-rapamycin.

In another embodiment the present invention relates to methods of prevention or treatment of cardiac and renal related conditions or diseases and arthritis or rheumatic arthritis related conditions or diseases by administration of a therapeutically effective amount of any preferred pharmaceutical composition according to the invention comprising an AT1 receptor blocker valsartan plus a mTOR inhibiting agent, e.g. rapamycin or a rapamycin derivative and a pharmaceutically acceptable carrier to a mammal in need thereof.

AT₁-receptor antagonists (also called angiotensin II receptor antagonists) are understood to be those active ingredients that bind to the AT₁-receptor subtype of angiotensin II receptor but do not result in activation of the receptor. As a consequence of the inhibition of the AT₁ receptor, these antagonists can, for example, be employed as antihypertensives or for treating congestive heart failure.

The class of AT₁ receptor antagonists comprises compounds having differing structural features, essentially preferred are the non-peptidic ones. For example, mention may be made of the compounds that are selected from the group consisting of valsartan (cf. EP 443983), losartan (cf. EP253310), candesartan (cf. 459136), eprosartan (cf. EP 403159), irbesartan (cf. EP45451 1), olmesartan (cf. EP 503785), tasosartan (cf. EP539086), telmisartan (cf. EP 522314), the compound with the designation E-1477 of the following formula

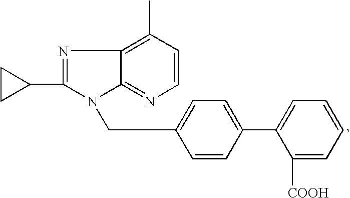

the compound with the designation SC-52458 of the following formula

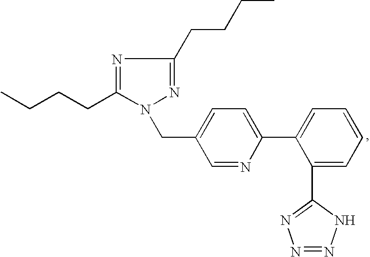

and the compound with the designation the compound ZD-8731 of the following formula

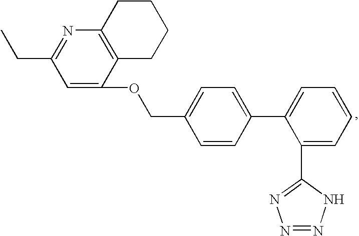

or, in each case, a pharmaceutically acceptable salt thereof.

Preferred AT₁-receptor antagonist are those agents that have been marketed, most preferred is valsartan or a pharmaceutically acceptable salt thereof.

Valsartan is the AT 1-receptor blocker (S)-N-(1-carboxy-2-methyl-prop-1-yl)-N-pentanoyl-N-[2;(1H-tetrazol-5-yl)biphenyl-4-yl-methyl]amine of formula (I)

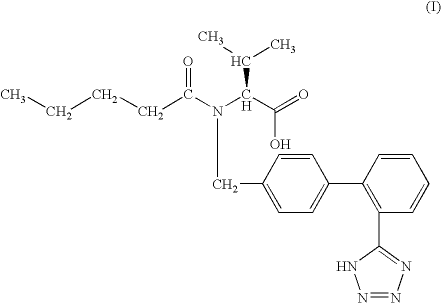

and is disclosed in EP 0443983 A and U.S. Pat. No. 5,399,578, the disclosures of which are incorporated herein in their entirety as if set forth herein.

The compounds to be combined can be present as pharmaceutically acceptable salts. If these compounds have, for example, at least one basic center, they can form acid addition salts. Corresponding acid addition salts can also be formed having, if desired, an additionally present basic center. The compounds having at least one acid group (for example COOH) can also form salts with bases. Corresponding internal salts may furthermore be formed, if a compound comprises, e.g., both a carboxy and an amino group. The corresponding active ingredient or a pharmaceutically acceptable salt thereof may also be used in form of a hydrate or include other solvents used for crystallization. Pharmacologically acceptable salts of the AT1 receptor blocker valsartan are preferably salts with bases, conveniently metal salts derived from groups Ia, Ib, IIa and IIb of the Periodic Table of the Elements, including alkali metal salts, e.g. potassium and especially sodium salts, or alkaline earth metal salts, preferably calcium or magnesium salts, and also ammonium salts with ammonia or organic amines.

Valsartan can be used in form of a crystalline, also partly crystalline and amorphous salts or salt hydrates.

Valsartan can also be used in form of solvates, such as hydrates, or in form of polymorphous forms of the salts.

A mTOR inhibitor is a compound which targets intracellular mTOR (“mammalian Target Of Rapamycin”). mTOR is a family member of phosphatidylinositol 3-kinase (PI3-kinase) related kinase. Rapamycin and rapamycin derivatives inhibit the mTOR pathway via a complex with its intracellular receptor FKBP12 (FK506-binding protein 12).

Rapamycin is a known macrolide antibiotic produced by Streptomyces hygroscopicus. By rapamycin derivative is meant a substituted rapamycin having mTOR inhibiting properties, e.g. rapamycin substituted in position 40 and/or 16 and/or 32, for example a compound of formula I

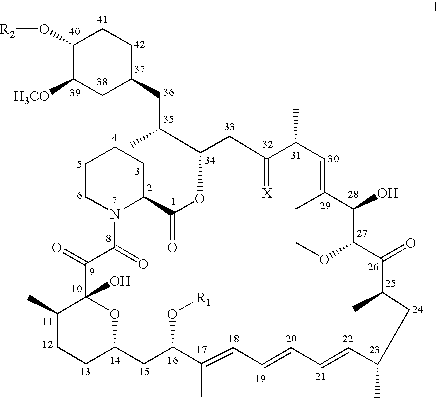

wherein
R₁ is CH₃ or C₃₋₆alkynyl,
R₂ is H, —CH₂—CH₂—OH, 3-hydroxy-2-(hydroxymethyl)-2-methyl-propanoyl or tetrazolyl, and

### X is ═O, (H,H) or (H,OH)

provided that R₂ is other than H when X is ═O and R₁ is CH₃,
or a prodrug thereof when R₂ is —CH₂—CH₂—OH, e.g. a physiologically hydrolysable ether thereof.

Representative rapamycin derivatives of formula I are e.g. 32-deoxorapamycin, 16-pent-2-ynyloxy-32-deoxorapamycin, 16-pent-2-ynyloxy-32(S or R)-dihydro-rapamycin, 16-pent-2-ynyloxy-32(S or R)-dihydro-40-O-(2-hydroxyethyl)-rapamycin, 40-[3-hydroxy-2-(hydroxymethyl)-2-methylpropanoate]-rapamycin (also called CC1779) or 40-epi-(tetrazolyl)-rapamycin (also called ABT578). A preferred compound is e.g. 40-0-(2-hydroxyethyl)-rapamycin disclosed in Example 8 in WO 94/09010 (referred hereinafter as Compound A), or 32-deoxorapamycin or 16-pent-2-ynyloxy-32(S)-dihydro-rapamycin as disclosed in WO 96/41807.

Rapamycin derivatives may also include the so-called rapalogs, e.g. as disclosed in WO 98/02441 and WO01/14387, e.g. AP23573, AP23464, AP23675 or AP23841.

Further examples of a rapamycin derivative are those disclosed under the name TAFA-93, biolimus-7 or biolimus-9.

It has surprisingly been found that the pharmaceutical combinations or compositions according to the invention can be used for the treatment or prevention of cardiac and renal related conditions or diseases, i e, selected from the group consisting of hypertension, heart failure such as (acute and chronic) congestive heart failure, left ventricular dysfunction and hypertrophic cardiomyopathy, diabetic cardiac myopathy, supraventricular and ventricular arrhythmias, atrial fibrillation, atrial flutter, detrimental vascular remodeling, myocardial infarction and its sequelae, atherosclerosis, angina (whether unstable or stable), renal insufficiency (diabetic and non- diabetic), angina pectoris, diabetes, secondary aldosteronism, primary and secondary pulmonary hypertension, renal failure conditions, such as diabetic nephropathy, glomerulonephritis, scleroderma, glomerular sclerosis, proteinuria of primary renal disease, and also renal vascular hypertension, diabetic retinopathy, the management of other vascular disorders, such as migraine, peripheral vascular disease, Raynaud's disease, luminal hyperplasia, cognitive dysfunction (such as Alzheimer's), glaucoma and stroke, and for the treatment or prevention of arthritis or rheumatic arthritis related conditions or diseases, i e, selected from the group consisting of, arthritis or rheumatic arthritis, e.g. including reduction of, alleviation of, stabilization of or relief from the symptoms or illness which affect the organism, particularly joints or vertebrae, including also slowing the progression (destruction of the joints) in moderate to severe rheumatoid arthritis, progressive, or erosive rheumatoid arthritis who had an inadequate response to treatment with disease-modifying antirheumatic drugs and the reduction of the side-effects (signs and symptoms) of arthritis or rheumatic arthritis.

It has surprisingly been found that, a combination of an AT1 receptor blocker, especially valsartan, and a mTOR inhibiting agent, e.g. rapamycin or a rapamycin derivative achieves greater therapeutic effect (a potentiation) than the administration of valsartan or the mTOR inhibiting agent alone. The combination surprisingly elicits an increased antihypertensive effect in rodent models of hypertension.

The combination also surprisingly ameliorates symptoms and improves mortality rates in animal models of heart failure.

Greater efficacy can also be documented as a prolonged duration of action. The duration of action can be monitored as either the time to return to baseline prior to the next dose or as the area under the curve (AUC) and is expressed as the product of the change in blood pressure in millimeters of mercury (change in mmHg) and the duration of the effect (minutes, hours or days).

Further benefits are that lower doses of the individual drugs to be combined according to the present invention can be used to reduce the dosage, for example, that the dosages need not only often be smaller but are also applied less frequently, or can be used to diminish the incidence of side effects.
Preferred are low dose combination of valsartan and mTOR inhibiting agent. The combined administration of an AT1 receptor blocker, especially valsartan, or a pharmaceutically acceptable salt thereof and a mTOR inhibiting agent results in a significant response in a greater percentage of treated patients, that is, a greater responder rate results, regardless of the underlying etiology of the condition. This is in accordance with the desires and requirements of the patients to be treated.
It can be shown that combination therapy with an AT1 receptor blocker, especially valsartan, and a mTOR inhibiting agent results in a more effective antihypertensive therapy through improved efficacy as well as a greater responder rate.
It can further be shown that an AT1 receptor blocker, especially valsartan, and mTOR inhibiting agent therapy proves to be beneficial in the treatment and prevention of myocardial infarction and its sequelae.
A valsartan plus mTOR inhibiting agent combination is also useful in treating renal failure conditions, such as diabetic nephropathy, glomerulonephritis, scieroderma, glomerular sclerosis, proteinuria of primary renal disease, and also renal vascular hypertension, diabetic retinopathy.

Thus in the present description the terms “treatment” or “treat” refer to both prophylactic or preventative treatment as well as curative or disease modifying treatment, including treatment of patients at risk of contracting the disease or suspected to have contracted the disease as well as patients who are ill or have been diagnosed as suffering from a disease or medical condition.

The Agents of the Invention, i.e. the mTOR inhibiting agent and an AT1 receptor blocker, especially valsartan, are preferably used in the form of pharmaceutical preparations that contain the relevant therapeutically effective amount of each active ingredient (either separately or in combination) optionally together with or in admixture with inorganic or organic, solid or liquid, pharmaceutically acceptable carriers which are suitable for administration. The Agents of the Invention may be present in the same pharmaceutical compositions, though are preferably in separate pharmaceutical compositions. Thus the active ingredients may be administered at the same time (e.g. simultaneously) or at different times (e.g. sequentially) and over different periods of time, which may be separate from one another or overlapping. The unit dose form may also be a fixed combination.

Preferably, the pharmaceutical compositions are adapted for oral or parenteral (especially oral) administration. Intravenous and oral, first and foremost oral, administration is considered to be of particular importance.

The pharmaceutical compositions according to the invention can be prepared in a manner known per se and are those suitable for enteral, such as oral, rectal, aerosol inhalation or nasal administration, and parenteral such as intravenous or subcutaneous administration, or compositions for transdermal administration (e.g. passive or iontophoretic) to mammals (warm-blooded animals), including man. Such compositions comprise a therapeutically effective amount of the pharmacologically active compound, alone or in combination with one or more pharmaceutically acceptable carriers, especially suitable for enteral or parenteral application. Typical oral formulations include tablets, capsules, syrups, elixirs and suspensions. Typical injectable formulations include solutions and suspensions. Tablets may be either film coated or enteric coated according to methods known in the art. Preferred are tablets and gelatin capsules comprising the active ingredient together with a) diluents, e.g. lactose, dextrose, sucrose, mannitol, sorbitol, cellulose and/or glycine; b) lubricants, e.g. silica, talcum, stearic acid, its magnesium or calcium salt and/or polyethyleneglycol; for tablets also c) binders e.g. magnesium aluminum silicate, starch paste, gelatin, tragacanth, methylcellulose, sodium carboxymethylcellulose and or polyvinylpyrrolidone; if desired d) disintegrants, e.g. starches, agar, alginic acid or its sodium salt, or effervescent mixtures; and/or e) absorbents, colorants, flavors and sweeteners. Injectable compositions are preferably aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1 to 85%, preferably about 1 to 70%, of the active ingredient.
The typical pharmaceutically acceptable carriers for use in the formulations described above are exemplified by: sugars such as lactose, sucrose, mannitol and sorbitol; starches such as cornstarch, tapioca starch and potato starch; cellulose and derivatives such as sodium carboxymethyl cellulose, ethyl cellulose and methyl cellulose; calcium phosphates such as dicalcium phosphate and tricalcium phosphate; sodium sulfate; calcium sulfate; polyvinylpyrrolidone; polyvinyl alcohol; stearic acid; alkaline earth metal stearates such as magnesium stearate and calcium stearate; stearic acid; vegetable oils such as peanut oil, cottonseed oil, sesame oil, olive oil and corn oil; non-ionic, cationic and anionic surfactants; ethylene glycol polymers; betacyclodextrin; fatty alcohols; and hydrolyzed cereal solids, as well as other non-toxic compatible fillers, binders, disintegrants, buffers, preservatives, antioxidants, lubricants, flavoring agents, and the like commonly used in pharmaceutical formulations.
Pharmaceutical preparations for enteral and parenteral administration are, for example, those in dosage unit forms, such as dragées, tablets or capsules and also ampoules. They are prepared in a manner known per se, for example by means of conventional mixing, granulating, confectioning, dissolving or lyophilising processes. For example, pharmaceutical preparations for oral administration can be obtained by combining the active ingredient with solid carriers, where appropriate granulating a resulting mixture, and processing the mixture or granulate, if desired or necessary after the addition of suitable adjuncts, into tablets or dragée cores.
Other orally administrable pharmaceutical preparations are dry-filled capsules made of gelatin, and also soft, sealed capsules made of gelatin and a plasticiser, such as glycerol or sorbitol. The dry-filled capsules may contain the active ingredient in the form of a granulate, for example in admixture with fillers, such as lactose, binders, such as starches, and/or glidants, such as talc or magnesium stearate, and, where appropriate, stabilisers. In soft capsules the active ingredient is preferably dissolved or suspended in suitable liquids, such as fatty oils, paraffin oil or liquid polyethylene glycols, it being possible also for stabilisers to be added.
Parenteral formulations are especially injectable fluids that are effective in various manners, such as intravenously, intramuscularly, intraperitoneally, intranasally, intradermally or subcutaneously. Such fluids are preferably isotonic aqueous solutions or suspensions which can be prepared before use, for example from lyophilised preparations which contain the active ingredient alone or together with a pharmaceutically acceptable carrier. The pharmaceutical preparations may be sterilised and/or contain adjuncts, for example preservatives, stabilisers, wetting agents and/or emulsifiers, solubilisers, salts for regulating the osmotic pressure and/or buffers.
Suitable formulations for transdermal application include an effective amount of a compound of the invention with carrier. Advantageous carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.
Suitable formulations for topical application, e.g. to the skin and eyes, include aqueous solutions, suspensions, ointments, creams, gels or sprayable formulations, for example, for delivery by aerosol or the like.
For example, the pharmaceutical preparations consist of from about 0.1-90%, preferably of from about 1% to about 80%, of the active compounds. Pharmaceutical preparations for enteral or parenteral administration are, for example, in unit dose forms, such as coated tablets, tablets, capsules or suppositories and also ampoules. These are prepared in a manner which is known per se, for example using conventional mixing, granulation, coating, solubulizing or lyophilizing processes. Thus, pharmaceutical preparations for oral use can be obtained by combining the active compounds with solid excipients, if desired granulating a mixture which has been obtained, and, if required or necessary, processing the mixture or granulate into tablets or coated tablet cores after having added suitable auxiliary substances. The dosage of the active compound can depend on a variety of factors, such as mode of administration, homeothermic species, age and/or individual condition.
Preferred dosages for the active ingredients of the pharmaceutical combination according to the present invention are therapeutically effective dosages, especially those which are commercially available.

Valsartan is supplied in the form of suitable dosage unit form, for example, a capsule or tablet, and comprising a therapeutically effective amount, e.g. from about 20 to about 320 mg, of valsartan which may be applied to patients. The application of the active ingredient may occur up to three times a day, starting, e.g., with a daily dose of 20 mg or 40 mg of valsartan, increasing via 80 mg daily and further to 160 mg daily up to 320 mg daily. Preferably, valsartan is applied once a day or twice a day in heart failure patients with a dose of 80 mg or 160 mg, respectively, each. Corresponding doses may be taken, for example, in the morning, at mid-day or in the evening. Preferred is q.d. or b.i.d. administration in heart failure.

Daily dosages for the mTOR inhibitor will, of course, vary depending on a variety of factors, for example the compound chosen, the particular condition to be treated and the desired effect. In general, however, satisfactory results are achieved on administration of the mTOR inhibitor at daily dosage rates of the order of ca. 0.01 to 5 mg/kg per day, particularly 0.5 to 5 mg/kg per day, as a single dose or in divided doses. A preferred daily dosage range is about from 0.1 to 30 mg as a single dose or in divided doses. The mTOR inhibitor, e.g. Compound A, may be administered by any conventional route, in particular enterally, e.g. orally, e.g. in the form of tablets, capsules, drink solutions or parenterally, e.g. in the form of injectable solutions or suspensions. Suitable unit dosage forms for oral administration comprise from ca. 0.05 to 15 mg active ingredient, usually 0.25 to 10 mg, e.g. Compound A, together with one or more pharmaceutically acceptable diluents or carriers therefore.

Rapamycin or derivatives thereof are well tolerated at dosages required for use in accordance with the present invention. For example, the NTEL for Compound A in a 4-week toxicity study is 0.5 mg/kg/day in rats and 1.5 mg/kg/day in monkeys.

The person skilled in the pertinent art is fully enabled to select a relevant test model to prove the efficacy of a combination of the present invention in the hereinbefore and hereinafter indicated therapeutic indications.

For example, representative studies are carried out with a combination of valsartan and a mTOR inhibiting agent, e.g., applying the following methodology:
Drug efficacy is assessed in various animal models including the spontaneously hypertensive rat (SHR) maintained on a normal, high or low salt diet.
Blood pressure will be monitored in the chronic study procedure by means of a radiotransmitter. The radiotransmitter is surgically implanted into the abdominal aorta of rats. Blood pressure is chronically monitored for periods of up 6 weeks.
Blood pressure, heart rate and activity are determined at various pre-selected time points before, during, and after drug administration. All measurements are performed in unrestrained and undisturbed animals. The maximum study time, determined by battery life, could be as long as nine months. For studies of this duration, rats are dosed orally (1-3 mL/kg vehicle), no more than twice daily or drug is administered via the drinking water or mixed with food. For studies of a longer duration, that is, up to 8 weeks, drugs are given via subcutaneously implanted osmotic minipumps. Osmotic minipumps are selected based on drug delivery rate and time. Valsartan dosages range from 1-50 mg/kg/day.
The hypertensive background of the SHR is modified either by chronic salt loading in an effort to suppress the renin angiotensin system (RAS) or chronic salt depletion to activate the RAS in the SHR. These manipulations will be carried out to more extensively evaluate the efficacy of the various test substances. Experiments performed in SHR are supplied by Taconic Farms, Germantown, N.Y. (Tac:N(SHR)fBR). A radiotelemetric device (Data Sciences International, Inc., St. Paul, Minn.) is implanted into the lower abdominal aorta of all test animals between the ages of 14 to 16 weeks of age. All SHR are allowed to recover from the surgical implantation procedure for at least 2 weeks prior to the initiation of the experiments. Cardiovascular parameters are continuously monitored via the radiotransmitter and transmitted to a receiver where the digitized signal is then collected and stored using a computerized data acquisition system. Blood pressure (mean arterial, systolic and diastolic pressure) and heart rate are monitored in conscious, freely moving and undisturbed SHR in their home cages. The arterial blood pressure and heart rate are measured every 10 minutes for 10 seconds and recorded. Data reported for each rat represent the mean values averaged over a 24-hour period and are made up of the 144-10 minute samples collected each day. The baseline values for blood pressure and heart rate consist of the average of three consecutive 24-hour readings taken prior to initiating the drug treatments. All rats are individually housed in a temperature and humidity controlled room and are maintained on a 12-hour light dark cycle.
Upon completion of the chronic studies, rats are anesthetized and the heart rapidly removed. After separation and removal of the atrial appendages, left ventricle and left plus right ventricle (total) are weighed and recorded. Left ventricular and total ventricular mass are then normalized to body weight and reported. All values reported for blood pressure and cardiac mass represent the group mean ±sem.
Vascular function and structure are evaluated after treatment to assess the beneficial effects of the combination. SHR are studied according to the methods described by Intengan et al., Circulation, Vol. 100, No. 22, pp. 2267-2275 (1999).

Myocardial infarction (MI) is produced in Sprague-Dawley rats by ligation of the left coronary artery. At four weeks after surgery, animals are treated with the combination of valsartan and cox-2 inhibitor. At 16 weeks after surgery, the animals are examined for hemodynamic function, euthanized and the hearts weighed. For assessments of hemodynamic function, rats are anesthetized with sodium pentobarbital (50 mg/kg i.p.). A miniature pressure transducer catheter (Millar Micro-Tip) is inserted into the right carotid artery and then advanced into the left ventricle. Left ventricular end-diastolic and left ventricular peak systolic pressures are recorded. After these assessments, the rats are sacrificed and the heart excised for weighing.

The available results indicate an unexpected therapeutic effect of a combination according to the invention.
The following examples illustrate the above-described invention; however, it is not intended to restrict the scope of this invention in any manner.

### EXAMPLES

Examples of valsartan formulation

### Formulation Example 1

### Film-Coated Tablets:

| | | |
|---|---|---|
| | Composition | |
| Components | Per Unit (mg) | Standards |

Granulation

| | | |
|---|---|---|
| Valsartan [=active ingredient] | 80.00 | |
| Microcrystalline cellulose/ | 54.00 | NF, Ph. Eur |
| Avicel PH 102 | | |
| Crospovidone | 20.00 | NF, Ph. Eur |
| Colloidal anhydrous silica/colloidal | 0.75 | Ph. Eur/NF |
| silicon dioxide/Aerosil 200 | | |
| Magnesium stearate | 2.5 | NF, Ph. Eur |

Blending

| | | |
|---|---|---|
| Colloidal anhydrous silica/colloidal | 0.75 | Ph. Eur/NF |
| silicon dioxide/Aerosil 200 | | |
| Magnesium stearate | 2.00 | NF, Ph. Eur |

Coating

| | |
|---|---|
| Purified water *) | — |
| DIOLACK pale red 00F34899 | 7.00 |
| TOTAL TABLET MASS | 167.00 |

*) Removed during processing.
The film-coated tablet is manufactured, e.g., as follows:
A mixture of valsartan, microcrystalline cellulose, crospovidone, part of the colloidal anhydrous silica/colloidal silicon dioxide/Aerosile 200, silicon dioxide and magnesium stearate is premixed in a diffusion mixer and then sieve through a screening mill. The resulting mixture is again pre-mixed in a diffusion mixer, compacted in a roller compactor and then sieve through a screening mill. To the resulting mixture, the rest of the colloidal anhydrous silica/colloidal silicon dioxide/Aerosile 200 are added and the final blend is made in a diffusion mixer. The whole mixture is compressed in a rotary tabletting machine and the tablets are coated with a film by using Diolack pale red in a perforated pan.

### Formulation Example 2

Film-coated tablets:

| | | |
|---|---|---|
| | Composition | |
| Components | Per Unit (mg) | Standards |

Granulation

| | | |
|---|---|---|
| Valsartan [=active ingredient] | 160.00 | |
| Microcrystalline cellulose/ | 108.00 | NF, Ph. Eur |
| Avicel PH 102 | | |
| Crospovidone | 40.00 | NF, Ph. Eur |
| Colloidal anhydrous silica/colloidal | 1.50 | Ph. Eur/NF |
| silicon dioxide/Aerosil 200 | | |
| Magnesium stearate | 5.00 | NF, Ph. Eur |

Blending

| | | |
|---|---|---|
| Colloidal anhydrous silica/colloidal | 1.50 | Ph. Eur/NF |
| silicon dioxide/Aerosil 200 | | |
| Magnesium stearate | 4.00 | NF, Ph. Eur |

Coating

| | |
|---|---|
| Opadry Light Brown 00F33172 | 10.00 |
| TOTAL TABLET MASS | 330.00 |

The film-coated tablet is manufactured, e.g., as described in Formulation Example 1.

### Formulation Example 3

### Film-Coated Tablets:

| | | |
|---|---|---|
| | Composition | |
| Components | Per Unit (mg) | Standards |

Core: Internal phase

| | | |
|---|---|---|
| Valsartan [=active ingredient] | 40.00 | |
| Silica, colloidal anhydrous (Colloidal | 1.00 | Ph. Eur, USP/NF |
| silicon dioxide) [=Glidant] | | |
| Magnesium stearate [=Lubricant] | 2.00 | USP/NF |
| Crospovidone [Disintegrant] | 20.00 | Ph. Eur |
| Microcrystalline cellulose [=Binding | 124.00 | USP/NF |
| agent] | | |

External phase

| | | |
|---|---|---|
| Silica, colloidal anhydrous, (Colloidal | 1.00 | Ph. Eur, USP/NF |
| silicon dioxide) [=Glidant] | | |
| Magnesium stearate [Lubricant] | 2.00 | USP/NF |

Film coating

| | |
|---|---|
| Opadry ® brown OOF 16711*) | 9.40 |
| Purified Water**) | — |
| TOTAL TABLET MASS | 199.44 |

*)The composition of the Opadry ® brown OOF16711 coloring agent is tabulated below.
**)Removed during processing

### Opadry® Composition:

| | Approximate % |
|---|---|
| Ingredient | Composition |
| Iron oxide, black (C.I. No. 77499, E 172) | 0.50 |
| Iron oxide, brown (C.I. No. 77499, E 172 | 0.50 |
| Iron oxide, red (C.I. No. 77491, E 172) | 0.50 |
| Iron oxide, yellow (C.I. No. 77492, E 172) | 0.50 |
| Macrogolum (Ph. Eur) | 4.00 |
| Titanium dioxide (C.I. No. 77891, E 171) | 14.00 |
| Hypromellose (Ph. Eur) | 80.00 |

The film-coated tablet is manufactured, e.g., as described in Formulation Example 1.

### Formulation Example 4

Capsules:

Composition
Per Unit (mg)

Components

| | |
|---|---|
| Valsartan [=active ingredient] | 80.00 |
| Microcrystalline cellulose | 25.10 |
| Crospovidone | 13.00 |
| Povidone | 12.50 |
| Magnesium stearate | 1.30 |
| Sodium lauryl sulphate | 0.60 |

Shell

| | |
|---|---|
| Iron oxide, red (C.I. No. 77491, EC No. E 172) | 0.123 |
| Iron oxide, yellow (C.I. No. 77492, EC No. E 172) | 0.123 |
| Iron oxide, black (C.I. No. 77499, EC No. E 172) | 0.245 |
| Titanium dioxide | 1.540 |
| Gelatin | 74.969 |
| TOTAL TABLET MASS | 209.50 |

The tablet is manufactured, e.g., as follows:
Granulation/Drying
Valsartan and microcrystallin cellulose are spray-granulated in a fluidized bed granulator with a granulating solution consisting of povidone and sodium lauryl sulphate dissolved in purified water. The granulate obtained is dried in a fluidized bed dryer.
Milling/Blending
The dried granulate is milled together with crospovidone and magnesium stearate. The mass is then blended in a conical srew type mixer for approximately 10 minutes.
Encapsulation
The empty hard gelatin capsules are filled with the blended bulk granules under controlled temperature and humidity conditions. The filed capsules are dedusted, visually inspected, weightchecked and quarantined until by Quality assurance department.

### Formulation Example 5

### Capsules:

Composition
Per Unit (mg)

Components

| | |
|---|---|
| Valsartan [=active ingredient] | 160.00 |
| Microcrystalline cellulose | 50.20 |
| Crospovidone | 26.00 |
| Povidone | 25.00 |
| Magnesium stearate | 2.60 |
| Sodium lauryl sulphate | 1.20 |

Shell

| | |
|---|---|
| Iron oxide, red (C.I. No. 77491, EC No. E 172) | 0.123 |
| Iron oxide, yellow (C.I. No. 77492, EC No. E 172) | 0.123 |
| Iron oxide, black (C.I. No. 77499, EC No. E 172) | 0.245 |
| Titanium dioxide | 1.540 |
| Gelatin | 74.969 |
| TOTAL TABLET MASS | 342.00 |

The formulation is manufactured, e.g., as described in Formulation Example 4.

### Formulation Example 6

### Hard Gelatine Capsule:

| Components | Composition Per Unit (mg) |
|---|---|
| Valsartan [=active ingredient] | 80.00 |
| Sodium laurylsulphate | 0.60 |
| Magnesium stearate | 1.30 |
| Povidone | 12.50 |
| Crospovidone | 13.00 |
| Microcrystalline cellulose | 21.10 |
| TOTAL TABLET MASS | 130.00 |

### Formulation Example 7

A hard gelatin capsule, comprising as active ingredient, e.g., (S)-N-(1-carboxy-2-methylprop-1-yl)-N-pentanoyl-N-[2′(1H-tetrazol-5-yl)biphenyl-4-yl-methyl]amine, can be formulated, for example, as follows:

### Composition:

| | | |
|---|---|---|
| (1) valsartan | 80.0 | mg |
| (2) microcrystalline cellulose | 110.0 | mg |
| (3) polyvidone K30 | 45.2 | mg |
| (4) sodium lauryl sulfate | 1.2 | mg |
| (5) crospovidone | 26.0 | mg |
| (6) magnesium stearate | 2.6 | mg |

Components (1) and (2) are granulated with a solution of components (3) and (4) in water. The components (5) and (6) are added to the dry granulate and the mixture is filled into size 1 hard gelatin capsules.

All publications and patents mentioned herein are incorporate by reference in their entirety as if set forth in full herein.

### Formulation Example 8

### Valsartan Compressed Solid Oral Dosage Form

### Formula

| valsartan | 80.0 | mg (40%) |
|---|---|---|
| AEROSIL 200 | 10.0 | mg (5%) |
| L-HPC* L-11 | 87.0 | mg (43%) |
| Magnesium Stearate | 3.0 | mg (1.5%) |
| AVICEL PH-301 | 10.0 | mg (5%) |
| L-HPC* L-21 | 5.0 | mg (2.5%) |
| AEROSIL 200 | 1.0 | mg (0.5%) |
| Talc | 2.0 | mg (1.0%) |
| Magnesium Stearate | 2.0 | mg (1.0%) |
| | 200.0 | mg |

*hydroxypropyl cellulose

### Method

The components (above the line) are blended in a container mixer. The blended material is sieved and pre-blended for an additional period of time in a container mixer. The blended material is compacted using a roller compactor (Bepex Pharmapaktor L 200/50 P, Hosokawa Micron Group) by applying a compaction force of 25-65 kN and a roller speed of 1.3-7.5 rpm. The compacted material is sieved again and the components below the line are added and final blended in a container mixer. Then 200 mg of the homogenous mixture is compressed into tablets using ovaloid punches (10×5.2 mm). The tablets obtained have a diameter of 8.5 mm and a thickness of 3.9 mm.

### Example 8A

### Film Coating

| | |
|---|---|
| Titanium dioxide | 1.00 mg |
| TC-5R* | 3.68 mg |
| PEG 6000 | 0.66 mg |
| Talc | 2.66 mg |
| | 8.00 mg |

*= hydroxypropylmethyl cellulose.

### Method

The PEG and cellulose are dissolved in the deionised water. The remaining components are suspended in the resulting solution. A spray coater apparatus (Dria-coater DRC-500, Powrex Ltd) is charged with the solid oral dosage form of Example 8A. The coating formulation is sprayed into the solid oral dosage form rotating in the apparatus at 6-12 rpm. Spray pressure is 1.9-2.2 Kg/cm² and the spray rate is 5.9-7.9 g/min.

Thereafter the coated solid oral dosage form is dried in the coater apparatus at a temperature of 40° C. until a moisture content in the coated solid oral dosage form is less than 2.5% by weight.

The coated tablet has a diameter of 8.6 mm and a thickness of 4.0 mm.

What is claimed is:

1. A pharmaceutical combination comprising an AT1 receptor blocker, or pharmaceutically acceptable salts thereof and a mTOR inhibiting agent, and optionally a pharmaceutically acceptable carrier.

2. Pharmaceutical combination according to claim 1, wherein the AT1-receptor blocker is valsartan and the mTOR inhibiting agent is selected from the group consisting of 32-deoxorapamycin, 16-pent-2-ynyloxy-32-deoxorapamycin, 16-pent-2-ynyloxy-32(S or R)-dihydro-rapamycin, 16-pent-2-ynyloxy-32(S or R)-dihydro-40-O-(2-hydroxyethyl)-rapamycin, 40-[3-hydroxy-2-(hydroxymethyl)-2-methylpropanoate]-rapamycin (also called CC1779), 40-epi-(tetrazolyl)-rapamycin (also called ABT578), 40-0-(2-hydroxyethyl) -rapamycin, 32-deoxorapamycin and 16-pent-2-ynyloxy-32(S)-dihydro-rapamycin and optionally a pharmaceutically acceptable carrier for simultaneous, sequential or separate use.

3. Pharmaceutical combination according to claim 1 wherein the mTOR inhibiting agent is 40-0-(2-hydroxyethyl)-rapamycin.

4. A method for treating cardiac and renal related conditions and arthritis or rheumatic arthritis related conditions comprising administering to a patient the pharmaceutical combination of claim 1.

5. The method according to claim 4, wherein the cardiac and renal related conditions or diseases are selected from the group consisting of hypertension, heart failure such as (acute and chronic) congestive heart failure, left ventricular dysfunction and hypertrophic cardiomyopathy, diabetic cardiac myopathy, supraventricular and ventricular arrhythmias, atrial fibrillation, atrial flutter, detrimental vascular remodeling, myocardial infarction and its sequelae, atherosclerosis, angina (whether unstable or stable), renal insufficiency (diabetic and non-diabetic), angina pectoris, diabetes, secondary aldosteronism, primary and secondary pulmonary hypertension, renal failure conditions, such as diabetic nephropathy, glomerulonephritis, scleroderma, glomerular sclerosis, proteinuria of primary renal disease, and also renal vascular hypertension, diabetic retinopathy, the management of other vascular disorders, such as migraine, peripheral vascular disease, Raynaud's disease, luminal hyperplasia, cognitive dysfunction (such as Alzheimer's), glaucoma and stroke, and the arthritis or rheumatic arthritis related conditions or diseases are selected from the group consisting of, illness affecting joints or vertebrates and side effects of arthritis or rheumatic arthritis.

6. (canceled)

7. (canceled)

8. A kit comprising in separate containers in a single package pharmaceutical compositions comprising in one container a pharmaceutical composition comprising a mTOR inhibiting agent, and in a second container a pharmaceutical composition comprising an AT 1-receptor blocker.

9. Kit according to claim 8, wherein the AT 1-receptor blocker is valsartan or a pharmaceutically acceptable salt thereof together with instructions for use in combination with a mTOR inhibiting agent.

10. (canceled)

11. (canceled)

12. (canceled)

13. (canceled)
